(19) United States
(12) Patent Application Publication
(10) Pub. No.: US 2006/0094760 A1
(43) Pub. Date: May 4, 2006

(54) Composition, system and method of treatment of gastrointestinal disorders with nizatidine oral solution

(76) Inventors: Abdel A. Fawzy, Easton, PA (US); George Bobotas, Tarpon Springs, FL (US); Keith S. Rotenberg, Danville, NJ (US)

Correspondence Address: Ann-Louise Kerner, Ph.D.;Wilmer Cutler Pickering Hale and Dorr LLP, 60 State Street, Boston, MA, 02109, US

(21) Appl. No.: 11/048,051

(22) Filed: Feb. 2, 2005

Related U.S. Application Data
(60) Provisional application No. 60/624,526, filed on Nov. 4, 2004.

(51) Int. Cl. A61K 31/445

(52) U.S. Cl. 514/327

(57) ABSTRACT

An alcohol-free, oral solution of nizatidine treats gastric and intestinal disorders. Oral doses of solution, which are equivalent to 150 mg twice daily, or 300 mg once daily, pill form of conventional nizatidine are orally administered and have a bioequivalency greater than 70%. The oral solution allows a wider population to obtain nizatidine treatment, particularly children, and the elderly, who have difficulty ingesting pills, can take the oral solution. Also, adolescents and younger children, in particular, can be treated with an alcohol-free oral solution.

This application claims the benefit of U.S. Provisional Patent Application Ser. No. 60/624,526 of George Bobotas and Abdel Fawzy, titled “System and Method of Treatment with Nizatidine Oral Solution” filed Nov. 4, 2004. The entirety of the provisional patent application is incorporated herein by reference.

### FIELD OF THE INVENTION

The present invention relates to a method of treating humans with an oral solution of nizatidine.

### BACKGROUND OF THE INVENTION

There are a number of medicines used to treat stomach or intestinal disorders. One of the most important of these is a group of medicines known as H₂-receptor antagonists or H₂ antagonists. The most common H₂ antagonists are cimetidine (TAGAMET®), ranitidine (ZANTAC®), famotidine (PEPCID®) and nizatidine (AXID®). These drugs are typically orally administered to treat and prevent ulcers in the stomach and the intestines.

Nizatidine basically works by decreasing the amount of acid the stomach produces. Accordingly, nizatidine is generally used to treat conditions in which the stomach produces too much acid and conditions in which acid comes up into the esophagus and causes heartburn, such as gastroesophageal reflux disease (GERD). Nizatidine has also been used to treat hyperacidity and Zollinger-Ellison Syndrome, which is a condition where large amounts of acid pour from the stomach.

Heartburn is the most common symptom of GERD, but regurgitation, difficulty in swallowing, chronic cough, hoarseness and a feeling of a lump in the throat may be associated symptoms. Studies indicate that as many as 45% of otherwise healthy people experience heartburn at least twice a month and 7% experience heartburn daily. Heartburn is often associated with meal-stimulated gastric acid secretion and gastroesophageal reflux with a pH of 4 or higher. Stomach acid is produced as a normal part of the digestive process. However, if large amounts of stomach acid are produced, the excess acid may flow back into the food pipe (esophagus), causing pain and a burning sensation known as heartburn. Excess stomach acid can also irritate the lining of the stomach and duodenum. This lining normally resists attack from the stomach acid, but if the lining is damaged, for example due to treatment with non-steroidal anti-inflammatory drugs (NSAIDs, such as ibuprofen), or large amounts of stomach acid are produced, a peptic ulcer can develop.

Nizatidine is generally a water-soluble competitive, reversible inhibitor of histamine at the histamine H₂-receptors, particularly those in the gastric parietal cells. Generally, as nizatidine blocks the H₂ receptors on the cells in the stomach, nizatidine prevents histamine from binding to the cells of the stomach. This prevention thereby decreases the amount of stomach acid produced by stomach cells and lowers the amount of acid in the stomach and duodenum. This action helps to relieve the pain of heartburn, and helps ulcers to heal, including those caused by NSAIDs. By decreasing the production of stomach acid, nizatidine can also be used to prevent ulcers from recurring.

The chemical formula for nizatidine is N-[2-[[[2-[(Dimethylamino) methyl]-4-thia-zolyl]methyl]thio]ethyl]-N′-methyl-2-nitro-1,1-ethenediamine. Nizatidine has the molecular formula C₁₂H₂₁N₅O₂S₂ representing a molecular weight of 331.46. The structural formula is as follows:

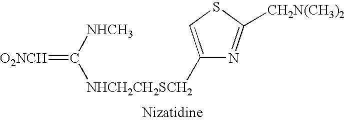

Generally, nizatidine, as well as the related drugs cimetidine, ranitidine and famotidine, are administered in solid form, such as tablets and capsules (commonly referred to herein as “pills” or “solids”). The pill forms of these drugs pose at least two major drawbacks: 1) difficulty in swallowing and 2) time to be broken down into effective particles to combat gastric secretions.

It is generally known in the art that many people have difficulty swallowing and ingesting tablets and capsules. It is believed that currently 26% of the total population has difficulty in swallowing pills. This percentage is considered to be higher in the pediatric patient population. Often, this difficulty leads to non-compliance with recommended medical therapy involving tablets and capsules. As a result, medical therapy would be rendered ineffective. It has been observed that the issue of non-compliance due to difficulty swallowing and ingesting pills is most pronounced in children and elderly populations. Thus, there is a significant problem with conventional nizatidine therapy in these populations.

One potential method of overcoming this drawback is to solubilize the pill form of the active ingredient into a liquid. However, forming a solution in this manner is known to significantly reduce the effectiveness of nizatidine. For example, studies in which nizatidine capsules are dissolved in apple juice results in a 27% reduction of nizatidine bioavailability. Thus, while there is a significant need for a non-pill form of nizatidine, it appears that a solution form of nizatidine is not as simple as dissolving a pill in a liquid.

The pill form of nizatidine is generally broken down into smaller particles before affecting gastric secretions. Accordingly, a certain amount of time must transpire while the pills solubilize and before the nizatidine is able to affect gastric secretions. In contrast, liquid suspensions more rapidly and effectively solubilize and have a greater ability to react with and neutralize gastric acid than tablets and capsules. However, as noted before, a liquid form of nizatidine, with the same bioequivalency as the pill form, is not conventionally available.

Oral solutions of cimetidine and ranitidine are available. However, a significant drawback related to these formulations is the presence of alcohol as a necessary component (2.8% with respect to cimetidine oral solution and 7.5% with respect to ranitidine syrup). It is preferable that certain populations, children and the elderly, in particular, avoid even modest alcohol intake. Thus, oral solutions of H₂ antagonists having a component of alcohol are not preferred, due to the limited applicability in these populations.

There is an unmet need in the art for nizatidine therapy involving a non-solid form of nizatidine to treat gastric and intestinal disorders. More specifically, there is an unmet need in the art to provide patients with difficulty or an aversion to swallowing pills an alternative method of nizatidine treatment. There is also an unmet need in the art to provide a method of nizatidine treatment that is able to solubilize faster than conventional pill forms of nizatidine. Moreover, there is an unmet need in the art to provide an oral solution formulation of H₂ antagonist without alcohol.

### SUMMARY OF THE INVENTION

The present invention overcomes the above-mentioned problems, as well as others, by administration of a safe and effective amount of nizatidine in an oral solution form to treat gastric and intestinal disorders. In some embodiments, the present invention is directed to orally administering to humans a therapeutically effective amount of a liquid or aqueous solution form of nizatidine. In some embodiments, the bioequivalency of a dose of oral solution nizatidine (e.g., 10 ml) of the present invention is similar to a dose of the conventional pill form of nizatidine (e.g., 150 mg). Due to the ease of administration, the oral solution of nizatidine will provide treatment benefits to a wider range of patients, particularly those who have difficulty swallowing tablets, such as the elderly and children. Moreover, the oral solution of nizatidine has a faster effect on gastric secretions than either the pill form of nizatidine or the dissolved pill in liquid form of nizatidine. The oral solution form of nizatidine does not require breakdown in the stomach before becoming effective. Thus, the oral solution is immediately effective upon ingestion and is therefore more responsive to gastric and intestinal disorders.

In some embodiments, the oral solution form of nizatidine is alcohol-free. The bioequivalency of the alcohol-free form of nizatidine treatment is similar to that of the conventional pill form. The removal of alcohol from the conventional formulation of H₂ antagonist allows nizatidine administration in groups who generally cannot or are should not consume the slightest amount of alcohol. For example, alcohol-free nizatidine is applicable for children, i.e., newborn to less than 18 years of age, for example 12 years and older. Accordingly, in some embodiments, the present invention is directed to orally administering to humans a therapeutically effective amount of oral solution nizatidine that is substantially free, or preferably entirely free, of alcohol.

In additional embodiments, the present invention is directed to a composition for treating gastrointestinal disorders in a human, the composition including a therapeutically effective amount of nizatidine comprised in an aqueous solution that is substantially alcohol-free. Preferred embodiments of the present invention include an oral solution comprising nizatidine which may include one or more, and preferably all of, the inactive ingredients methylparaben, propylparaben, glycerin, sodium alginate, purified water, sodium chloride, saccharin sodium, sodium citrate dihydrate, citric acid anhydrous, sucrose, bubble gum flavor, artificial sweetness enhancer, and sodium hydroxide.

Other features of the present invention will become apparent to those skilled in the art upon examination of the following or upon learning by practice of the invention.

### DESCRIPTION OF THE PREFERRED EMBODIMENTS

The present invention provides a safe and effective oral administration of nizatidine to treat gastric and intestinal disorders. In particular, the present invention provides an oral solution for the administration of nizatidine. The oral solution is preferably at least as effective as administration of nizatidine in pill form in treating gastrointestinal disorders, such as GERD, heartburn impaired gastric motility and peptic ulcers, specifically inhibiting gastric secretions to an extent not before seen in the art.

In one variation, oral solution nizatidine significantly inhibited nocturnal gastric acid secretion for up to 12 hours. In another variation, oral solution nizatidine inhibited gastric secretion stimulated by food, caffeine, betazole, and pentagastrin. The present invention also is useful in minimizing weight gain, or promoting weight loss, in subjects engaged in a dietary regimen.

The oral solution is indicated for up to eight weeks for the treatment of active duodenal ulcer. With treatment, in most patients, the ulcer will heal within four weeks. Moreover, the oral solution is indicated for maintenance therapy for duodenal ulcer patients at a reduced dosage. Accordingly, the dosage may be broken down into two parts: 1) a period of a full daily dosage, and 2) a period of a reduced daily dosage. The oral solution is indicated for up to twelve weeks for the treatment of endoscopically diagnosed esophagitis, including erosive and ulcerative esophagitis, and associated heartburn due to GERD. The oral solution is indicated for up to eight weeks for the treatment of active benign gastric ulcer. In pediatric patients, the oral solution is indicated for ages twelve years and older. The oral solution is indicated for up to eight weeks for the treatment of endoscopically diagnosed esophagitis, including erosive and ulcerative esophagitis, and associated heartburn due to GERD.

The present invention is an aqueous pharmaceutical solution comprising nizatidine. Nizatidine can be present, in some embodiments, at a concentration of between about 5 mg/ml and about 45 mg/ml, preferably between about 10 mg/ml and about 30 mg/ml, and most preferably between about 15 mg/ml and about 20 mg/ml. The pH of the formulation is preferably greater than 3, preferably greater than 4, and more preferably between about 5 and about 8. Some embodiments of the oral solution formulation include at least a pharmaceutically acceptable preservative and at least a pharmaceutically acceptable chelating agent. The oral solution is also preferably substantially alcohol-free, most preferably entirely free, of ethanol.

In one variation, the equivalence of about 150 to 450 mg, preferably about 300 mg capsular nizatidine (e.g. about 10 to 30 ml, preferably about 20 ml oral solution nizatidine), is administered once per day. In another variation, nizatidine is administered twice daily. Each administration is preferably equivalent to about 60 to 300 mg, preferably about 150 mg, capsular nizatidine (e.g. about 4 to 20 ml, preferably about 10 ml, oral solution nizatidine).

In one embodiment, the first administration of nizatidine is taken prior to consumption of a meal, which is likely to induce gastric secretions. Accordingly, the nizatidine is first administered about 15 to about 90 minutes before the meal is consumed by the user. Preferably, the administration of nizatidine occurs about 15-45 minutes, and more preferably 30 minutes before the meal. In alternate embodiments, the administration of nizatidine is in the evening, e.g., generally between 6 pm and 12 midnight, preferably just prior to bedtime, e.g., 9 pm, 10 pm, 11 pm, etc.

The second administration of nizatidine is generally taken by the user after the meal. In one variation, the second administration is taken immediately after the meal. In another variation, the second administration is taken between about 30 minutes after the meal to about 180 minutes after the meal. Preferably, the second administration is taken about 60 minutes to about 120 minutes after the meal. Alternatively, if the meal is consumed in the evening, the second administration may be taken immediately prior to overnight sleep, i.e., at bedtime, e.g., 9 pm, 10 pm, 11 pm, etc. It is generally known in the art that nizatidine effectively inhibits nocturnal gastric acid secretions. In yet another variation, the second administration is taken in the evening, which is generally defined as the period between 6 pm and 12 midnight.

Like the tablet or capsule formulation, the oral solution of nizatidine is a safe and effective treatment for gastric and intestinal disorders. In fact, the bioequivalency of nizatidine through the oral solution is similar to that of the conventional pill form. Each 1 ml preferably contains about 15-20 mg of nizatidine. Thus, about 10 ml of the oral solution is equivalent to about a 150-200 mg capsule of nizatidine (interchangeably referred to herein as “solid nizatidine”). More preferably, about 10 ml of the oral solution nizatidine is bioequivalent to about 150 mg of the pill form of nizatidine.

A preferred embodiment of the present invention includes a clear, yellow, oral solution with bubble gum flavor, wherein each 1 ml contains 15 mg of nizatidine. The solution may include the inactive ingredients methylparaben, propylparaben, glycerin, sodium alginate, purified water, sodium chloride, saccharin sodium, sodium citrate dihydrate, citric acid anhydrous, sucrose, bubble gum flavor, artificial sweetness enhancer, and sodium hydroxide. This embodiment of the present invention is marketed as Axid OS® or Axid Oral Solution® by Reliant Pharmaceuticals, Inc., Liberty Corner, N.J.

The oral solution of the present invention is preferably administered in the following manner. For active duodenal ulcers, the oral dosage of oral solution nizatidine for adults may be equivalent to 150 mg to 450 mg solid nizatidine, preferably about 200 mg to 400 mg solid nizatidine, and most preferably about 300 mg solid nizatidine, once daily at bedtime. An alternative dosage regimen is half these amounts, twice daily. For maintenance of healed duodenal ulcers, the oral dosage of oral solution nizatidine for adults may be equivalent to 75 mg to 225 mg solid nizatidine, preferably about 150 mg, once daily at bedtime. For treatment of GERD, the oral dosage in adults for the treatment of erosions, ulcerations, and associated heartburn, may be equivalent to 5 mg to 225 mg solid nizatidine, preferably about 150 mg, twice daily. For treatment of active benign gastric ulcers, the oral solution dosage may be equivalent to 150 mg to 450 mg solid nizatidine, preferably about 200 mg to 400 mg, and most preferably about 300 mg, given either twice daily or once daily, preferably at bedtime.

The following is the preferred pediatric dosing. For pediatric patients under 12 years of age, the preferred dosage is equivalent to 10 mg to 300 mg solid nizatidine, twice daily. More preferably, the preferred dosage is 75 mg, twice daily. For pediatric patients 12 years of age and older, the preferred dosage is equivalent to 75 mg to 300 mg solid nizatidine, preferably about 150 mg, twice daily. In another variation, pediatric dosing is calculated per weight of the treated subject. The pediatric dosing of oral solution nizatidine is equivalent to 2.5 mg/kg to 5.0 mg/kg, twice daily.

The oral solution delivery system of the present invention can comprise a combination of active and non-active pharmaceutical ingredients (also known generally herein as “excipients”). Non-active ingredients, for example, serve to solubilize, suspend, emulsify, stabilize, preserve, protect, color, flavor, and fashion the active ingredients into an applicable and efficacious preparation that is safe, convenient, and otherwise acceptable for use. The active ingredient, i.e., nizatidine or a suitably acceptable salt thereof, can constitute about 0.1 to 25% of the total weight percent of the delivery system, for example.

Generally, excipients and non-active ingredients of orally administered nizatidine are of any type generally known in the art. Compositions intended for oral use may contain one or more agents, such as sweetening agents, flavoring agents, coloring agents and the like, in order to provide a pharmaceutically elegant and palatable preparation. Syrups and elixirs may be formulated with suitable sweetening agents, for example, glycerol, sorbitol, or sucrose. Such formulations may also contain suitable demulcents, preservatives and flavoring and coloring agents.

The sweetening agent is likely to be beneficial for pediatric and adolescent compliance of nizatidine medical therapy. The sweetening agent may be an artificial sweetener, such as aspartame, or a natural sweetener, such as a sugar such as sucrose or sorbitol. The presence of the sweetener, especially sorbitol, has been found to enhance the stability of the formulation. The presence of the sweetener helps to increase patient compliance in that it masks the unpleasant taste of the nizatidine. The importance of patient compliance cannot be overemphasized particularly with young children who are highly likely to reject unpalatable medicines. Sorbitol is preferred as the sweetener. It is preferred to have the formulation contain 10 to 50% w/w of the sweetener, more preferably from 25 to 45% w/w of the sweetener, and desirably about 35% w/w of the sweetener, e.g. sorbitol. The sweetener may comprise a mixture of sugars, e.g., sorbitol and sucrose.

Minor amounts of other ingredients such as tonicity agents (e.g. NaCl), pH adjusters (e.g., a base such as NaOH, acids such as citric), emulsifiers or dispersing agents, buffering agents, preservatives, wetting agents, thickening agents (e.g. polyvinyl alcohol) and gelling agents (e.g. polaxamer) may also be present. Particularly, preferred compositions contain sufficient amounts of the foregoing and/or other ingredients to be a substantially isotonic and/or buffered to a physiologically acceptable pH.

The preservative may be any pharmaceutically acceptable preservative that is compatible with the formulation, e.g. sodium benzoate or an alkyl hydroxybenzoate such as propyl- or preferably methyl-hydroxybenzoate. A sufficient preservative should be present to maintain the solution in a sterile condition, and in general the solution may contain up to about 0.1% w/w, e.g. from 0.01 to 0.08% w/w, of the preservative.

In one more variation of the present invention, the oral use formulation is formulated by admixing nizatidine with excipients, including suspending agents (e.g., sodium carboxymethyl cellulose, methyl cellulose, hydroxy propyl methyl cellulose, sodium alginate, polyvinylpyrrolidone, gum tragacanth and gum acacia) and wetting agents (e.g., lecithin, polyoxyethylene stearate, heptadecaethyleneoxycetanol, polyoxyethylene sorbitol monooleate, or polyoxyethelyne sobirtan monooleate). The aqueous suspensions may also contain one or more suitable preservatives (e.g., ethyl, or n-propyl, p-hydroxy benzoate), one or more suitable coloring agents, one or more suitable flavoring agents and one or more suitable sweetening agents (e.g., sucrose, saccharin, or sodium or calcium cyclamate). The oral solution of the present invention is prepared by completely dissolving the nizatidine, and optionally the excipients, in solution. The process for manufacturing the oral solution will be understood by one skilled in the art.

The oral bioavailability of nizatidine exceeds 50%, and preferably exceeds 60%, and more preferably exceeds 70%. Peak plasma concentrations (e.g., 700 to 1,800 μg/L for a 150-mg dose and 1,400 to 3,600 μg/L for a 300-mg dose) preferably occur from 0.5 to 3 hours following the dose.

Table 1 presents pharmacokinetic data of nizatidine administered orally to adolescents with gastroesophageal reflux (GER) and healthy adults. Pharmacokinetic parameters for adolescent patients ages 12 to 18 years are comparable to those obtained for adults.

TABLE 1
Pharmokinetics of oral solution nizatidine

| | | | Cₘₐₓ | Tₘₐₓ | AUC_{0-∞} | CL_{F} | vd_{F} | T_{1/2} |
|---|---|---|---|---|---|---|---|---|
| Age Range | Formulation | Dose | (ng/mL) | (h) | (ng · h/mL) | (L/h) | (L) | (h) |
| 12-18 yr | Capsule | 150 mg SD | 1422.9 | 1. [symbol] | [symbol] | 41.0 | 71.4 | 1.2 |
| Adolescents | | 150 mg SS | 1480.2 | 1.4 | [symbol] 776.1 | 41.1 | 74.2 | 1.3 |
| with GER | | | | | | | | |
| Healthy | Capsule | 150 mg SD | 1 [symbol] 7. [symbol] | 1.0 | 3703.1 | 41.9 | [symbol] 3.4 | 1.4 |
| Adults | Oral Solution | 150 mg SD | 1 [symbol] 0. [symbol] | 0. [symbol] | [symbol] 610.9 | 43.0 | [symbol] .4 | 1.4 |
| | Apple Juice | 150 mg SD | 7 [symbol] 2. [symbol] | 1. [symbol] | 2694.1 | 57.5 | 142.3 | 1.7 |

SD—single dose
SS—steady state
Administration of nizatidine capsules in apple juice results in 27% reduction of nizatidine bioavailability.

For the purposes of this application, the “oral solution” and the “aqueous solution” of nizatidine of this invention are not bioequivalent to solid nizatidine dissolved in a liquid. It is notable in Table 1 that the formulation of the oral solution of the present invention has properties distinguishable from capsules dissolved in liquids, e.g., apple juice. For example, the Cₘₐₓ and AUC are significantly less in capsules dissolved in apple juice as compared to the oral solution of the present invention. In fact, in one variation of the present invention, administration of nizatidine capsules in apple juice results in approximately a 27% reduction of nizatidine bioavailability as compared to solid nizatidine and oral solution nizatidine of the present invention. Thus, the oral solution of the present invention is far different from dissolving a capsule of nizatidine in liquid.

The oral solution form of nizatidine provides a numbers of benefits previously unmet by the conventional administration of nizatidine. For example, some segments of the population have difficulty ingesting pills, thereby making medicinal therapies in pill form inapplicable, ineffective, and generally difficult to follow. For this population, conventional pill-form nizatidine has been unavailable, thereby marginalizing this population from effective treatment of gastric or intestinal disorders. The oral solution of nizatidine remedies this problem. The oral solution of the present invention facilitates administration of nizatidine in subjects who cannot or choose not to ingest pill forms. The oral solution of nizatidine therefore has a wider application than conventional nizatidine.

Moreover, the conventional liquid form of H₂ antagonist incorporates an amount of alcohol. Accordingly, conventional H₂ antagonist therapy has been limited to populations who are able to ingest alcohol. The oral solution of nizatidine, in accordance with embodiments of the present invention, can provide an alcohol-free formulation of nizatidine that effectively expands the target population and the scope of H₂ antagonist treatment. For example, the oral solution of nizatidine can be given to populations, such as pediatrics, from newborns to under 18 years of age, who are generally kept away, or should be kept away, from alcohol-based products and therapies.

Embodiments of the invention have now been described in accordance with the above advantages. It will be appreciated that these examples are merely illustrative of the invention. Many variations and modifications will be apparent to those skilled in the art.

What we claim:

1. A method for treating gastrointestinal disorders in a human, the method comprising:
administering a therapeutically effective amount of an oral solution comprising nizatidine, wherein the oral solution is substantially alcohol-free.

2. The method of claim 1, wherein 1 ml of the oral solution is bioequivalent to 13.5-17.5 mg of nizatidine in pill form.

3. The method of claim 1, wherein 1 ml of the oral solution is bioequivalent to about 15 mg nizatidine in pill form.

4. The method of claim 1, wherein the oral solution is entirely free of alcohol.

5. The method claim 1, wherein the human is at least 12 years old.

6. The method of claim 2, wherein the human is administered from about 3.5 ml to about 22.5 ml daily dose of the oral solution.

7. The method of claim 6, wherein the human is administered an about 5 ml, 10 ml, 15 ml or 20 ml daily dose of the oral solution.

8. The method of claim 1, wherein the administration occurs once daily.

9. The method of claim 8, wherein the administration is from about 17.5 ml to about 22.5 ml of the oral solution.

10. The method of claim 9, wherein the administration is about 20 ml of the oral solution.

11. The method of claim 8, wherein the administration occurs in the evening.

12. The method of claim 8, wherein the administration occurs at bedtime.

13. The method of claim 1, wherein the administration occurs twice daily.

14. The method of claim 13, wherein each administration is from about 7.5 ml to about 12.5 ml of the oral solution.

15. The method of claim 14, wherein each administration is about 10 ml of the oral solution.

16. The method of claim 13, wherein a first daily administration is prior to a meal and a second daily administration is after the meal.

17. The method of claim 16, wherein the first daily administration is from immediately to about 90 minutes prior to the meal.

18. The method of claim 16, wherein the second daily administration is immediately after the meal.

19. The method of claim 16, wherein the second daily administration is from immediately to about 180 minutes after the meal.

20. The method of claim 19, wherein the second daily administration is from immediately to about 120 minutes after the meal.

21. The method of claim 16, wherein the second daily administration is at bedtime and the meal is consumed in the evening.

22. The method of claim 16, wherein the second daily administration is in the evening.

23. The method of claim 1, wherein the administration is repeated at least once daily for a treatment period ranging from about 2 days to about 12 weeks.

24. The method of claim 23, wherein a first part of the treatment period comprises a first full daily dosage of oral solution and a second part of the treatment period comprises a second daily dosage of oral solution, wherein the second daily dosage is reduced as compared to the first daily dosage.

25. The method of claim 1, wherein the gastrointestinal disorders are selected from the group consisting of esophagitis, gastroesphogeal reflux disease, hyperacidity, Zollinger-Ellison Syndrome, heartburn, duodenal ulcer, and gastric ulcer.

26. The method of claim 1, wherein a human having an age ranging from newborn to less than 12 years old is administered a daily dose ranging from about 0.6 ml to about 22.5 ml of the oral solution.

27. The method of claim 1, wherein a human having an age ranging from newborn to less than 12 years old is administered a daily dose ranging from about 0.16 ml/kg body weight to about 0.33 ml/kg body weight of the oral solution.

28. The method of claim 1, wherein the administration of the oral solution provides a Cₘₐₓ greater than 1300 ng/ml and/or an AUC greater than 3500 ng×H/ml.

29. The method of claim 1, wherein the administration of the oral solution provides a peak plasma concentration ranging from 700 to 1,800 g/L for a 10 mL dose from about 0.5 to about 3 hours after administration.

30. The method of claim 1, wherein the administration of the oral solution provides a peak plasma concentration ranging from 1,400 to 3,600 g/L for a 20 mL dose from about 0.5 to about 3 hours after administration.

31. A composition for treating gastrointestinal disorders in a human, the composition comprising a therapeutically effective amount of an oral solution comprising nizatidine, wherein the oral solution comprising the nizatidine is substantially alcohol-free.

32. The composition according to claim 31, wherein the composition further comprises one or more pharmaceutically acceptable excipients.

33. The composition according to claim 32, wherein the excipients are one or more members selected from the group consisting of a sweetening agent, a flavoring agent, a coloring agent, a tonicity agent, a preservative, a suspending agent, an emulsifier, a dispersing agent, a buffering agent, a wetting agent, a thickening agents, and a gelling agent.
